(19) United States
(12) Patent Application Publication
(10) Pub. No.: US 2006/0015068 A1
(43) Pub. Date: Jan. 19, 2006

(54) Intravenous catheter assembly

(76) Inventors: Shai Amisar, Tel Aviv (IL); Ronen Radomski, Haifa (IL); Paul Froom, D. N Menashe (IL)

Correspondence Address: NIXON & VANDERHYE, PC, 901 NORTH GLEBE ROAD, 11TH FLOOR, ARLINGTON, VA, 22203, US

(21) Appl. No.: 10/509,829

(22) PCT Filed: Mar. 27, 2003
(86) PCT No.: PCT/IL03/00256

§ 371 Date: Aug. 5, 2005

(30) Foreign Application Priority Data
Apr. 4, 2002 (IL) 148994

(51) Int. Cl. A61M 5/178

(52) U.S. Cl. 604/164.01; 604/171

(57) ABSTRACT

A self-contained sterile catheter apparatus for use with an intravenous cannula element. The cannula element has first and second ends and a bore formed therebetween, and is configured for transcutaneous positioning such that the first end is adapted to protrude from a limb of a subject and the second end is brought into communication with an interior of a body organ of a subject. The self-contained sterile catheter apparatus includes first and second ends and a flexible catheter tube therebetween, the catheter tube having a predetermined length and a diameter adapted for slidable insertion through the bore of the intravenous cannula element into the subject, and an integral sterile environment containment element thereby to allow insertion of the catheter tube through the cannula element into the subject in a generally non-sterile environment.

### FIELD OF THE INVENTION

The present invention relates, generally to intravenous catheters and, more specifically, to flexible intravenous catheters.

### BACKGROUND OF THE INVENTION

It is known in the art to provide peripheral intravenous therapy using a catheter having a short cannula or catheter tube to provide access into subcutaneous veins thereby to introduce medication, drugs, chemotherapy, nutrition and various other fluids into a vein of a subject. The present procedure includes inserting a hypodermic needle together with a catheter having a cannula into a suitable vein site, withdrawing the needle and leaving the catheter cannula in the vein. Such a catheter is provided with a suitable closure and various adapter mechanisms to enable the introduction of fluid medicaments from a hypodermic syringe or from an intravenous drip.

Studies over the past thirty years have shown that, up to seventy percent of subjects receiving peripheral intravenous therapy, develop an inflammatory reaction to the vein known as phlebitis. These studies include:
Maki D G, Goldman D A, Rhame F S: “Infection control in intravenous therapy” Ann Intern Med 1973; 79:876-87;
Turnidge J: “Hazards of peripheral intravenous lines” Med J Aust 1984; 141:37-40;
Lewis G B, Hecker J F: “Infusion thrombophlebitis” Br J Anaesth 1985; 57:22-33;
Hessov I: “Prevention of infusion thrombophlebitis” Acta Anaesthesiol Scand Supple. 1985; 29:33-37;
Turen S J: “Infusion phlebitis: a review of the literature” Parenterals 1987; 14:37-40; and
Maki D G, Ringer M: “Risk factors for infusion-related phlebitis with small peripheral venous catheters”.

Phlebitis necessitates the removal of the cannula, reinsertion of a cannula into an alternative site and, often, local treatment and analgesic drugs. The extent of this problem is best understood by the fact that about fifteen percent of the general population is admitted annually into hospitals. Approximately seventy percent of admitted hospital subjects receive intravenous treatment. The majority of such subjects require treatment extending over three days or more. The incidence of phlebitis has been found to exceed fifty percent of all such subjects by the fourth day after cathetization and, in the case of subjects receiving intravenous antibiotics, the risk is doubled.

With these subjects, there are many risk factors influencing the incidence of phlebitis, including an increased risk in female subjects, the specific anatomic site of insertion and a previous history of phlebitis. In addition, structural parameters, such as the materials used in the manufacture of small catheters, add to the risk of phlebitis as described in Maki D G et al, above. Specifically the risk of infection increases with time and it is generally recommended that the catheter site be changed every three days.

Actual infection of the catheter end is not a common cause of phlebitis. The most common cause is chemical irritation, specifically in subjects having chemotherapy or peripheral intravenous nutrition. Such subjects generally have multiple treatments or long-term hyperosmolar fluid introduction. Subjects suffer pain as well as long-term damage or destruction of veins, making determining new insertion sites more problematic and sometimes frustrating and time-consuming for the medical professional.

There are several alternatives to peripheral intravenous therapy using a short catheter, namely:
Central venous catheter with or without the use of subcutaneous ports known as a Porta-cath,
Peripherally inserted central lines (PICC lines), and
Peripheral ports.

These alternatives are referred to in:
Schuman E, Ragsdale J: “Peripheral ports are a new option for central venous access” J Am Coll Surg 1995; 180:456-60;
Lyon R D, Griggs K A, Johnson A M, Olsen J R: “Long-term follow-up of upper extremity implanted venous access devices in oncology subjects” J Vasc Interv Radiol 1999; 10:463-71;
Jills J R, Cardella J F, Cardella K, Waybill P N: “Experience with 100 consecutive central venous access arm ports placed by interventional radiologists” J Vasc Interv Radiol 1997; 8:983-9;
Minassian V A, Sood A K, Lowe P, Sorosky J I, Al-Jurf A S, Buller R E: “Longterm central venous access in gynecologic cancer subjects” J Am Coll Surg 2000; 191:403-9; and in
Reynolds J V, Walsh K, Ruigrok J, Hyland J M: “Randomised comparison of silicone versus Teflon cannulas for peripheral intravenous nutrition” Ann R Coll Surg Engl 1995; 77:447-9.

It has recently been shown by Jills J R, et al, hereinabove, and in Pullyblank A M, Carey P D, Pearce S Z, Tanner A G, Guillou P J, Monson J R. Ann R. Coll Surg Engl 1994; 76:33-8, that peripheral access system ports have a longer life and a lower infection risk compared to a centrally placed catheter. Nonetheless, peripheral access system ports are found to be invasive, incur various complicating factors and have to be removed in the event of a fever developing.

PICC lines have been shown to be able to be left in place for longer periods of time but are relatively expensive. Furthermore, PICC lines are not widely used because the technique for insertion differs from that used most frequently by medical professionals. Insertion requires the placement of a primary catheter, and insertion of a line through the catheter followed by removal of the primary catheter. Longer catheters appear to reduce the phlebitis risk as described in
Linder L E, Wojciechowski J, Zachrisson B F, Curelaru I, Gustavsson B, Hultman E, Bylock A. “Half-way” venous catheters. IV. Clinical-experience and thrombogenicity Acta Anaesthesiol Scand Suppl 1985; 81:40-47, and in
Pearson M L. Guideline for prevention of intravascular device related infects. The Hospital Infection control practices advisory committee. Center for Disease Control and Prevention.

It has been repeatedly shown that use of a PCC line drastically reduces the incidence of phlebitis. However, much practice is needed to properly carry out an insertion. Moreover, in up to thirty two percent of cases, the PCC line procedure had to be repeated as a result of the appearance of phlebitis, or the clogging or tearing of the catheter.

Referring now to U.S. Pat. No. 5,704,919 to Menachem Kraus et al entitled “Intravenous Cannula Assembly” there is disclosed an intravenous assembly having a distal end insertable into a subject. There is provided a guide needle, which is moved into a required position with respect to a cannula such that the sharp extremity of the needle projects beyond the distal end of the cannula. A subcutaneous vein is pierced with this mechanism and the guide needle retracted via the proximal end of the cannula, from which it is removed into a protective enclosure, leaving the cannula positioned within the vein.

The cannula described in the foregoing patent is relatively short, the distal end being positioned within the vein relatively close to the insertion site. In the event that the patient requires extended intravenous therapy, it is necessary to re-insert a new cannula into an alternative subcutaneous vein site every few days to avoid phlebitis or thrombosis. This causes the patient additional trauma, requires additional time spent by the medical professional and necessitates having a sterile field of operation.

It is apparent that there is a need in the art to provide a solution to or an alleviation of the problems of phlebitis and thrombosis, caused by presently available techniques for intravenous therapy. The choice site generally selected by medical professionals is the cephalic vein in the forearm. It is large and accessible and any infusion set can be secured out of the way, making it less likely to be pulled out accidentally. Should relocation prove necessary, there exists only one other such choice site, leaving alternatives, which are less ideal. This is most especially the case where a number of vein entries are necessitated. There is a need in the art, therefore, to reduce or avoid the necessity for frequently relocating intravenous sites.

### SUMMARY OF THE INVENTION

The present invention aims to provide an improved intravenous catheter system, such that problems associated with current practice are avoided or minimized. Specifically, in accordance with current practice for patients having extended intravenous therapy, on average, it is required to reposition an intravenous catheter, every three days. Re-positioning a subcutaneous intravenous catheter is necessitated as a result of occurrence of phlebitis or thrombosis in the vein in the vicinity of the catheter tip. Other circumstances, such as a patient developing a fever, may also require the repositioning of a catheter.

The present invention relates to an improved intravenous catheter system including a multi-use entry-port element having first and second ends having a bore formed there-between. The entry-port element is configured for transcutaneous positioning such that the second end is brought into liquid flow communication with a vein of a subject. The system further includes a catheter having first and second ends and a flexible catheter-tube there-between, the catheter tube having a predetermined length and a diameter adapted for slidable insertion through the entry-port element into the vein of the subject.

The present invention also relates to a self-contained sterile catheter apparatus, for use with an intravenous cannula element having first and second ends and having a bore formed therebetween, the cannula element configured for transcutaneous positioning such that the first end is adapted to protrude from a limb of a subject and the second end is brought into communication with an interior of a body organ of a subject, the self-contained sterile catheter apparatus includes
first and second ends and a flexible catheter tube therebetween, the catheter tube having a predetermined length and a diameter adapted for slidable insertion through the bore of the intravenous cannula element into the body organ of the subject, and
an integral sterile environment containment element thereby to allow insertion of the catheter tube through the cannula element into the body organ of a subject in a generally non-sterile environment.

According to a preferred embodiment of the present invention, there is provided an improved intravenous catheter system in which the multi-use entry-port element includes
a hub having a slide adapter-connector fixably disposed at the first end of the entry-port element, thereby to provide sealed slidable access of the catheter-tube into the entry-port element and thereafter into the vein of the subject;
a cannula having an aperture formed at a second end thereof, the cannula fixably attached to the hub and disposed at the second end of the entry-port element, the cannula adapted for insertion into the vein of the subject; and
a removable needle, having first and second ends, a needle-hub attached at the first end, a sharp extremity at the second end and a length sufficient to extend through the entry-port element, the needle slidably housed in the entry-port, the sharp extremity projecting beyond the aperture at the second end of the cannula thereby to pierce through the skin and vein wall into the vein of the subject, and thereby to provide entry into the vein for the cannula.

According to another preferred embodiment of the present invention, there is provided an improved intravenous catheter system in which the catheter includes
a connector element disposed at the first end of the catheter and having a removable cap, the connector element configured to facilitate, in the absence of the cap, connection of an intravenous therapeutic device to the first end of the catheter; and
a slidable-connector element disposed at the second end of the catheter and having a removable cap, the slidable-connector element, configured to facilitate connection of the second end of the catheter to the first end of the entry-port element and thereby to facilitate sliding the catheter tube therethrough into the entry-port element and into the vein of the subject.

According to another preferred embodiment of the present invention, there is provided an improved intravenous catheter system also including an integral sterile environment containment element thereby to allow insertion of said catheter tube through said cannula element into the vein of a subject in a generally non-sterile environment. Furthermore, according to variations of the preferred embodiment of the present invention the integral sterile environment containment element includes a longitudinal disposable sheath configured to contain the catheter tube longitudinally therewithin or a cylindrical casing configured to contain the catheter tube as a withdrawable coil therewithin.

According to another variation of the preferred embodiment of the present invention the entry-port element has mounting lugs fixably disposed thereto, for securing the entry-port element to the subject.

According to a further variation of the preferred embodiment of the present invention the second end of the cannula is formed with a taper, thereby to provide a compression lip seal between the cannula aperture and the catheter tube.

According to additional variations of embodiments of the present invention the connector element and the slide adapter-connector configured at the first and second ends of the catheter, respectively, includes a Luer Lock.

According to another embodiment of the present invention, there is provided an improved intravenous catheter system in which a removable stiffener element is slidably disposed within the flexible catheter tube. This stiffener element increases the stiffness of the catheter tube, and aids insertion thereof through the entry-port element into the vein of the subject.

According to further embodiments of the present invention, there is provided an improved intravenous catheter system in which the entry-port element includes a selectably operable locking device for locking the catheter tube in a selected position with respect to a selected drug delivery location within the vein of the subject. Also, there is included a valve for regulating a flow of liquid through the catheter tube.

The present invention also relates to a self-contained sterile catheter apparatus, for use with an intravenous cannula element having first and second ends and having a bore formed therebetween. The cannula element is configured for transcutaneous positioning such that the first end is adapted to protrude from a limb of a subject and the second end is brought into communication with an interior of a body organ of a subject, the self-contained sterile catheter apparatus includes
first and second ends and a flexible catheter tube therebetween, the catheter tube having a predetermined length and a diameter adapted for slidable insertion through the bore of the intravenous cannula element into the body organ of the subject, and
an integral sterile environment containment element thereby to allow insertion of the catheter tube through the cannula element into the body organ of a subject in a generally non-sterile environment.

In accordance with an embodiment of the present invention, for use with an intravenous cannula element the catheter includes
a connector element disposed at said first end of said catheter and having a removable cap, said connector element configured to facilitate, in the absence of said cap, connection of an intravenous therapeutic device to said first end of said catheter; and
a slidable-connector element disposed at said second end of said catheter and having a removable cap, said slidable-connector element, configured to facilitate connection of said second end of said catheter to said first end of said entry-port element, thereby to facilitate sliding said catheter tube therethrough into said entry-port element and thereafter into the vein of the subject.

In accordance with another embodiment of the present invention, for use with an intravenous cannula element the integral sterile environment containment element is selected from the group, which consists of:
a longitudinal collapsible sheath configured to contain the catheter tube longitudinally therewithin; and
a cylindrical casing configured to contain the catheter tube as a withdrawable coil therewithin.

In accordance with a variation of the aforementioned embodiment of the present invention the cylindrical casing includes a clutch device thereby to control forceful insertion of the catheter tube through the entry port into a vein, so as to avoid damaging the vein wall.

In accordance with another embodiment of the present invention, there is a flexible catheter tube for use with an intravenous cannula element and also including a removable stiffener element slidably disposed within the flexible catheter tube. This has the effect of increasing the stiffness of the catheter tube, and thereby to aid insertion thereof through the entry-port element into the vein of the subject.

Furthermore, there is provided a method for introducing an improved intravenous catheter system into a subcutaneous vein of a subject. The method includes
introducing a multi-use entry-port, having a removable piercing-needle slidably housed therein, into an outer wall of a subcutaneous vein of a subject,
withdrawing the removable piercing-needle from the entry-port,
advancing the multi-use entry-port into the vein,
connecting a catheter device, having a flexible catheter tube, to the entry-port, and
slidably inserting the flexible catheter-tube through the entry-port into the subcutaneous vein of the subject.

According to a further embodiment of the present invention, the method includes the step of securing the entry-port element to the skin of the subject.

According to another embodiment of the present invention, withdrawing the removable piercing-needle from the entry-port includes partially withdrawing the removable piercing-needle from the entry-port after the vein wall has been pierced, to guard the sharp extremity of the piercing needle and to avoid transfixing the vein.

According to an additional embodiment of the present invention, when using a catheter tube having a removable stiffener element slidably disposed therein, the method includes slidably removing the stiffener element from the catheter.

An additional embodiment of the present invention provides for the repeated use of a catheter inserted through a multiple entry-port without having to repeatedly relocate the catheter vein-site every few days. Catheters of different lengths are used to avoid repetitive location of the catheter tip at the same location within the subcutaneous vein. Alternatively, there is provision for adjusting the position of a catheter end and locking the catheter tube in each new position. The problem of phlebitis is substantially reduced. Patients do not have the repeated trauma of having the catheter re-inserted into other vein sites. Thrombosis or other blockages are removed by placing another catheter into the entry-port. And the medical professional is able to carry out the changing of the catheter without the need for a sterile field. Once a multi-use entry-port is in position, inserting replacement catheters or adjusting the position of the catheter is possible, even in the most extreme circumstances.

### BRIEF DESCRIPTION OF THE DRAWINGS

The present invention will be more fully understood and its features and advantages will become apparent to those skilled in the art by reference to the ensuing description, taken in conjunction with the accompanying drawings, in which:

FIG. 1 is a schematic partial cross-sectional view illustrating insertion of a multi-use entry-port forming part of an intravenous catheter system into a vein of a subject, in accordance with a preferred embodiment of the present invention;

FIG. 2 is a schematic partial cross-sectional view illustrating withdrawal of the needle from the inserted multi-use entry-port of the catheter system of the present invention;

FIG. 3 is a schematic partial cross-sectional view illustrating a flexible catheter prior to connecting to the multi-use entry-port and insertion therein;

FIG. 3A, is a schematic isometric projection view of a flexible catheter including an integral sterile environment containment element prior to connecting to the multi-use entry-port;

FIG. 4 is a schematic partial cross-sectional view illustrating the flexible catheter connected to the multi-use entry-port prior to insertion through the multi-use entry-port prior to insertion into the vein of a subject;

FIG. 5 is a schematic partial cross-sectional view illustrating the flexible catheter inserted through the multi-use entry-port into the vein of a subject;

FIG. 6 is a schematic view showing alternative shapes of a second free end of a multi-use entry-port;

FIG. 7 is a schematic partial cross-sectional view of a second end of a multi-use entry-port with a catheter tube extending therethrough;

FIG. 8 is a schematic partial cross-sectional view illustrating use of a stiffening element within the flexible catheter;

FIG. 9 is a schematic partial cross-sectional view illustrating the flexible catheter, having a stiffening element, connected to the multi-use entry-port prior to insertion through the multi-use entry-port prior to insertion into the vein of a subject;

FIG. 10 is a schematic partial cross-sectional view illustrating the stiffening element within the flexible catheter, fully inserted through the multi-use entry-port into the vein of a subject;

FIG. 11 is a schematic partial cross-sectional view illustrating a flexible catheter tube inserted through the multi-use entry-port into the vein of a subject, the stiffening element withdrawn from the flexible catheter;

FIG. 12 is a schematic view of a multi-use entry-port having a locking device;

FIG. 13 is a schematic view of a multi-use entry-port having a flow control valve; and

FIG. 14 is a schematic view of a cylindrical receptacle for containing a coiled catheter tube in a sterile environment.

### DETAILED DESCRIPTION OF THE INVENTION

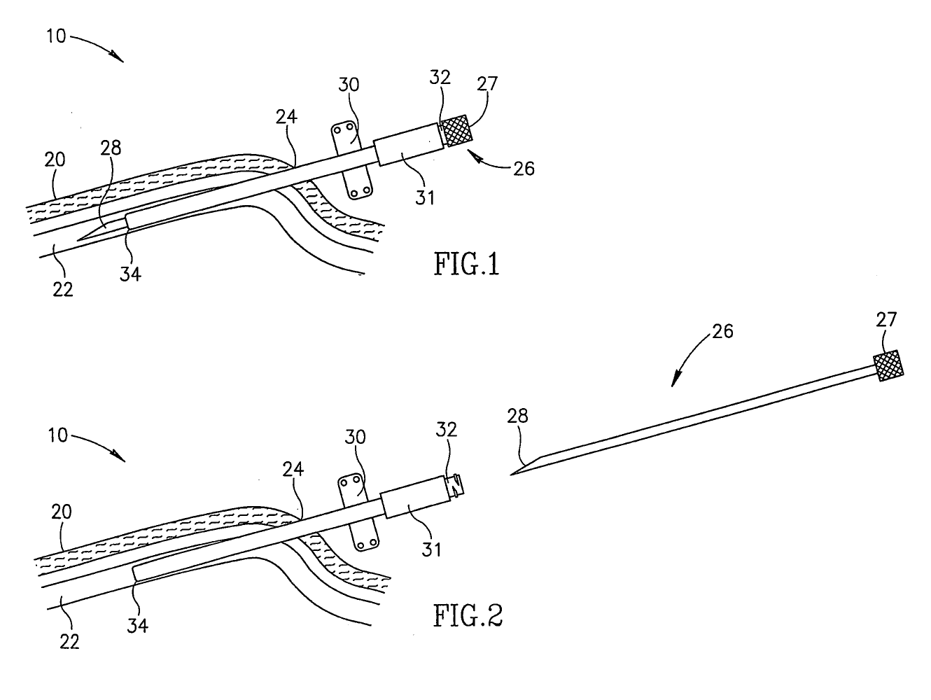

The present invention relates to an apparatus and a method whereby intravenous therapy is applied to a subject, such that the incidence of phlebitis or thrombosis in a vein of the subject is substantially reduced without necessitating a multiplicity of intravenous entries. This is generally achieved by the use of a short multi-use entry-port or a standard intracatheter disposed in a vein of the subject. Thereafter, a catheter having a flexible tube or cannula of a predetermined length, is attached thereto and slidably inserted therethrough into the vein of the subject. This procedure is sequentially illustrated in FIGS. 1 to 5 and described hereunder, in accordance with a preferred embodiment of the present invention. To reduce the risk of phlebitis or thrombosis, the catheter is periodically replaced with another of a different length, or the position of the catheter adjusted, without necessitating removal and relocation of the entry-port.

Referring to FIG. 1 there is seen a partial cross-sectional view of a skin surface referenced 20 and a subcutaneous vein-wall referenced 22. Also seen is a multi-use entry-port, generally referenced 10, subcutaneously introduced into vein 22. Entry-port 10 includes a cannula referenced 24, securing lugs referenced 30, a hub referenced 31, and a Luer Lock adapter referenced 32 attached thereto. To facilitate piercing of the skin 20 and vein-wall 22 and achieving entry thereto, by cannula 24, a needle generally referenced 26 is slidably positioned within multi-use entry-port 10. A sharp extremity referenced 28 of needle 26 protrudes from a second end referenced 34 of cannula 24 and, to retain needle 26 in this position, it is fastened to adapter 32 by a threaded needle hub referenced 27.

It is necessary to avoid transfixing the vein 22. After needle extremity 28 has pierced skin 20 and vein-wall 22, and cannula end referenced 34 has entered a short distance into vein-wall 22, needle 26 is partially withdrawn holding hub 27, such that extremity 28 is no longer exposed. Referring now to FIG. 2, there is seen multi-use entry-port 10, inserted through skin 20 and vein wall 22, having needle 26 slidably withdrawn therefrom.

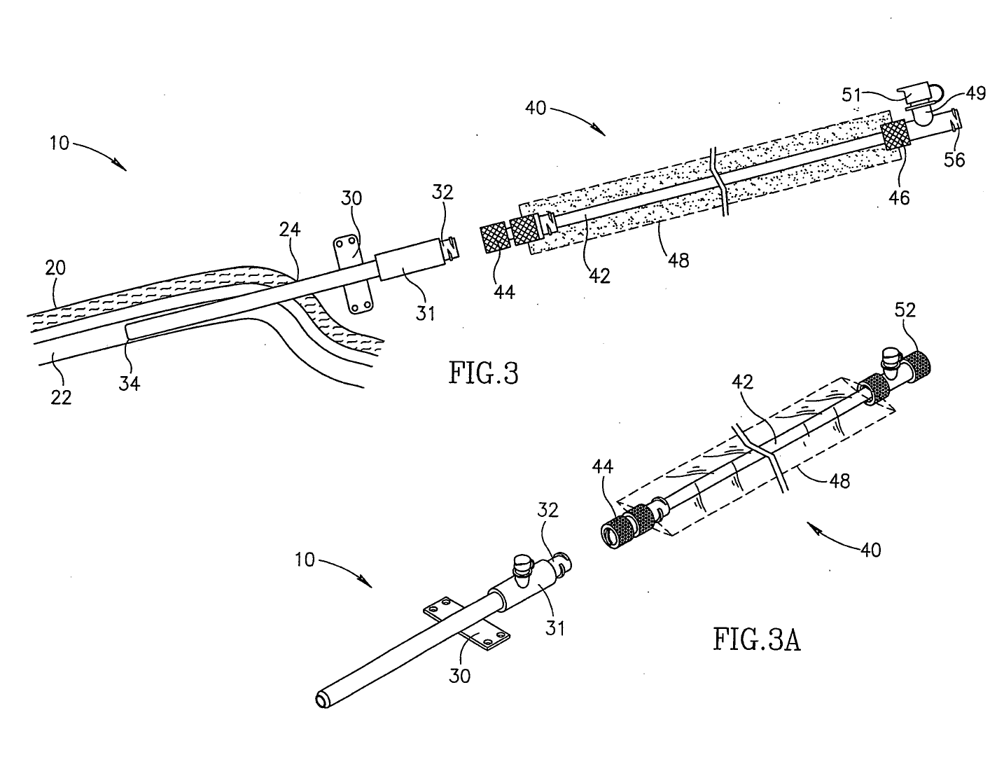

Thereafter, referring to FIG. 3, there is seen, in accordance with a preferred embodiment of the present invention, adjacent to entry port 10 and prior to connection thereto, a flexible catheter, generally referenced 40, having a flexible tube referenced 42 contained in an integral sterile sheath, referenced 48. Catheter 40 has a Luer Lock connector referenced 46 at a first end thereof. To facilitate dispensing of an additional medication, a second port referenced 49, having a removable cover cap referenced 51, is preferably formed at the first end of catheter 40. Referring now to FIG. 3A, there is seen an isometric projection view of catheter 40, having an integral sterile environment containment sheath, prior to connecting to the multi-use entry-port and entry-port 10, including a cover cap referenced 52 fastened over connector 46. A Luer Lock slide adapter-connector referenced 44 is seen at a second end of catheter 40 thereby to connect second end of catheter 40 to adapter 32 of entry-port 10.

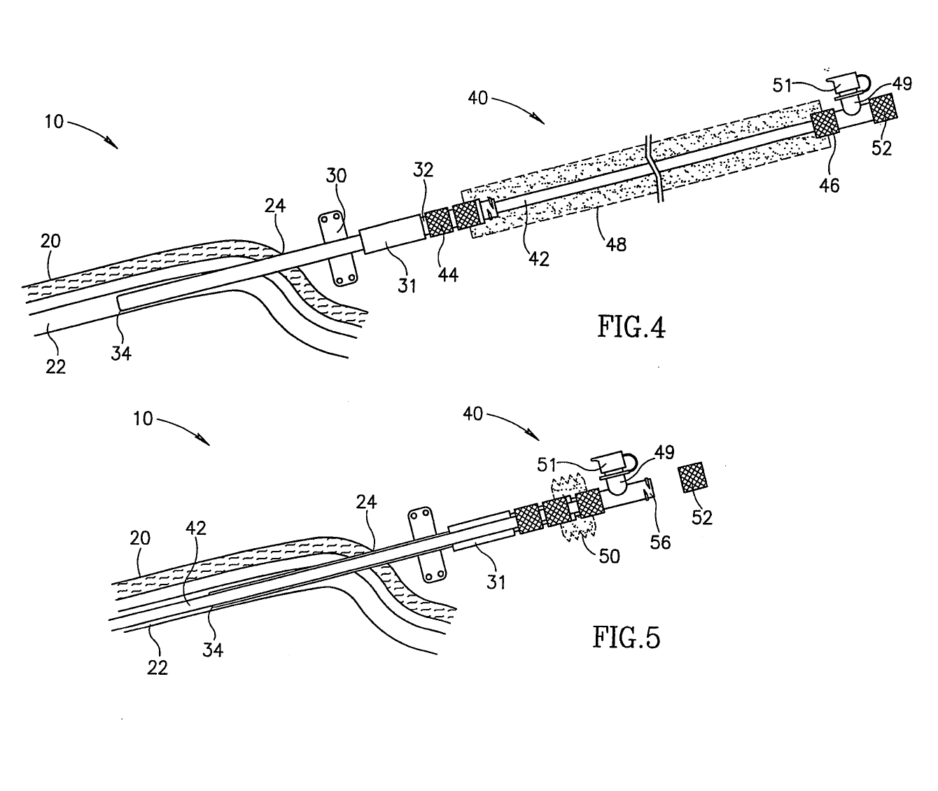

In order to facilitate insertion of catheter tube 42 through entry port 10 and into vein 22, and, referring now to FIG. 4, there is seen catheter 40 having Luer Lock slide adapter-connector 44 connected to Luer Lock adapter 32 of multi-use entry-port 10. Tube 42 is then slidably inserted through multi-use entry-port 10 into vein 22 of the subject.

Furthermore, referring to FIG. 5, there is seen flexible tube 42 in position in vein 22, after being slidably pushed through slide adapter-connector 44 into multi-use entry-port 10 and thereafter into and along vein 22. Luer Lock connector 46 is moved until it is adjacent to Luer Lock slide adapter-connector 44 and fixably attached thereto. Sterile sheath 48 is seen in a collapsed configuration, referenced 50. After removing cover cap 52, there is seen exposed a connector referenced 56, thereby to facilitate connecting thereto an intravenous therapy device (not shown) such as an intravenous drip or hypodermic syringe.

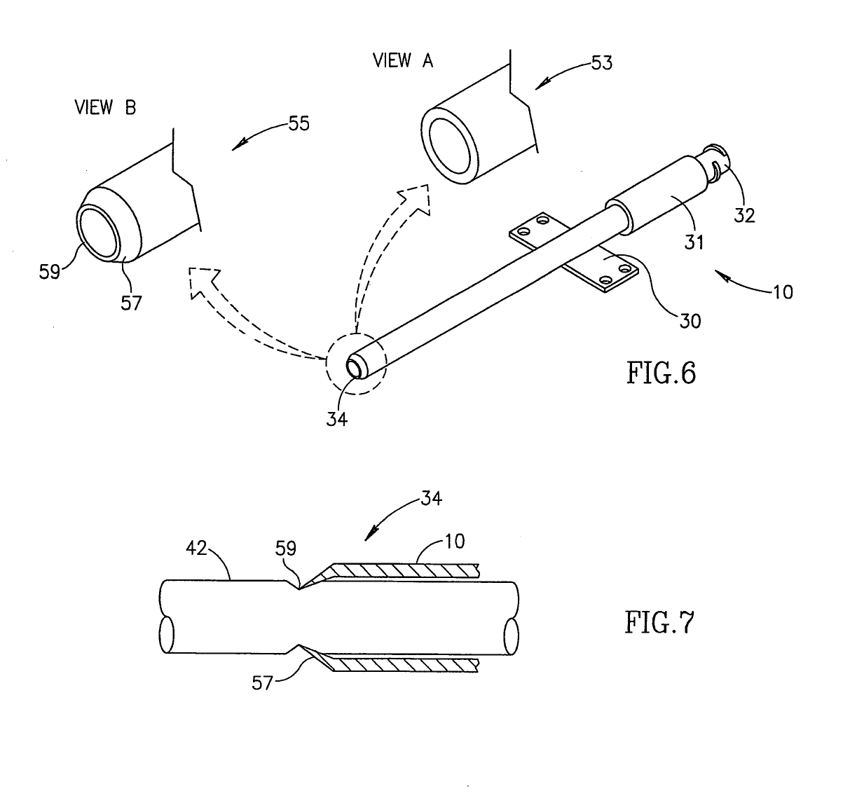

Referring now to FIG. 6, there is seen, in accordance with a further embodiment of the present invention, a schematic isometric view of entry-port 10 together with alternative configurations of end 34 thereof. View A illustrates a straight cylindrical edge, generally referenced 53. View B illustrates a tapered end generally referenced 55 having a tapered edge referenced 57 and an internal compression lip seal referenced 59. Referring now to FIG. 7, there is seen a partial cross-sectional view of end 34 of cannula 24 with catheter tube 42 passing therethrough. To achieve a seal between multi-use entry-port 10 and catheter flexible tube 42, as mentioned above, second end 34 of multi-use entry-port 10 has a taper 57, thereby to form an internal compression lip seal 59 against flexible tube 42.

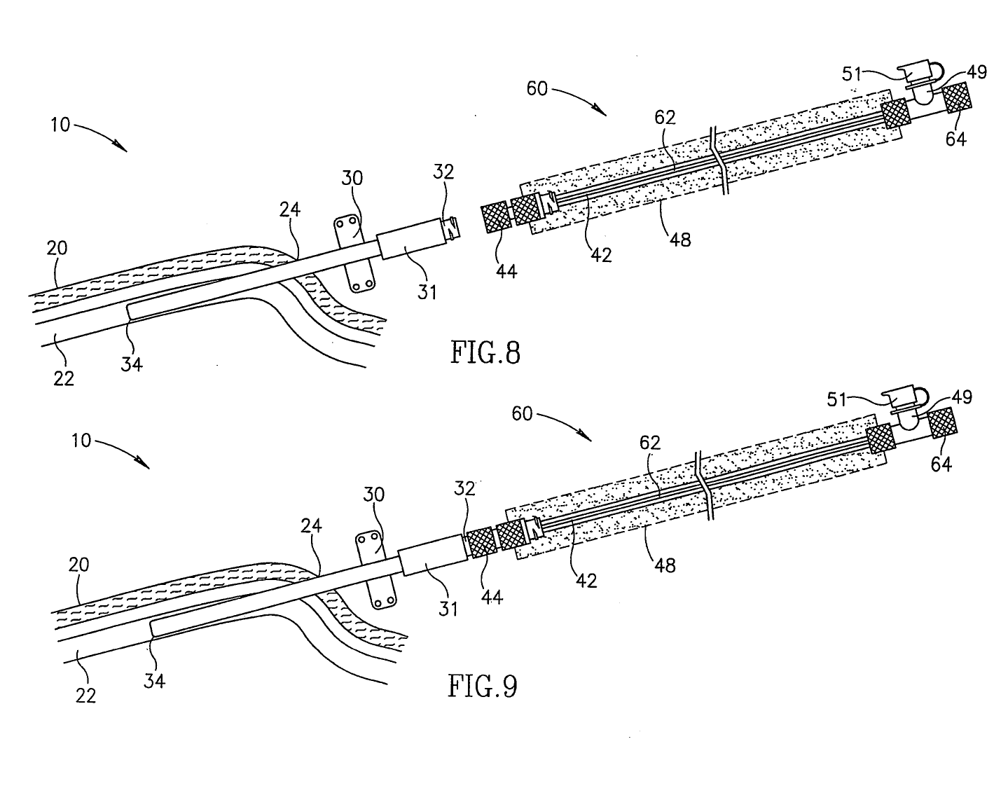

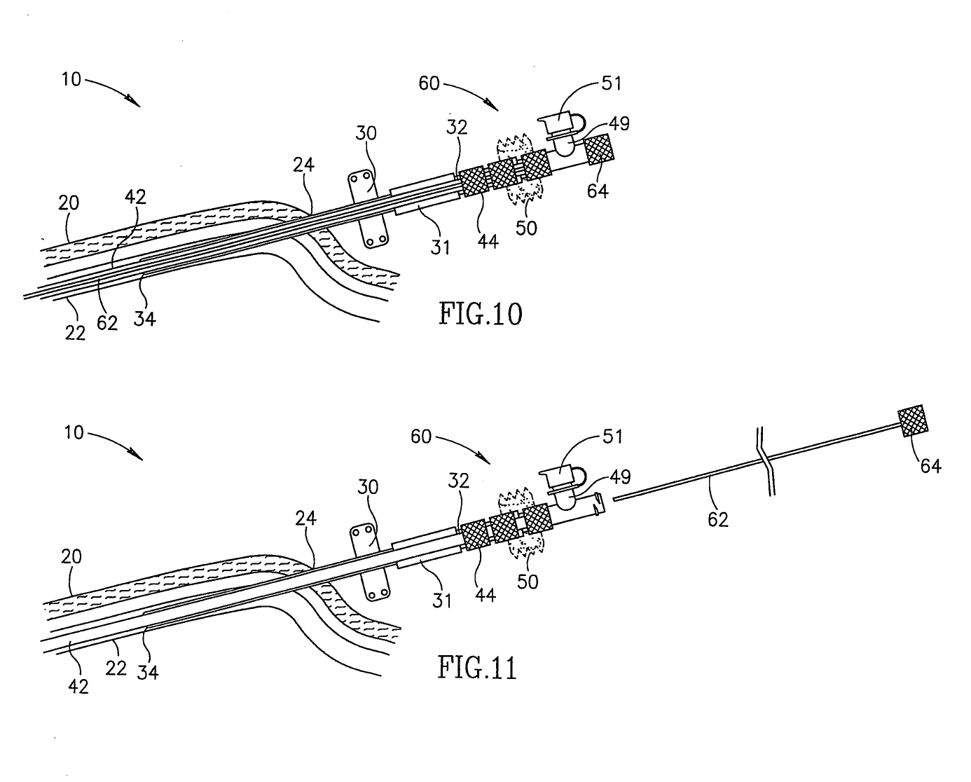

In accordance with an alternative embodiment of the present invention, for specific applications, it is necessary to provide a stiffening effect to tube 42 for insertion into vein 22. Introduction of a stiffened catheter tube 42 is sequentially illustrated in FIGS. 8 to 11. Referring now to FIG. 8, there is seen a catheter, generally referenced 60, substantially similar to that seen in FIGS. 1-7 hereinabove, including a stiffening element referenced 62 disposed slidably within flexible tube 42. A cover cap referenced 64 at a first end thereof, serves as a hub to stiffening element 62. Stiffening element 62 facilitates insertion of catheter flexible tube 42 into a subject's vein. Referring to FIG. 9 there is seen catheter 60 connected using connector 44 to entry-port 10 at adaptor 32. There is further seen in FIG. 10, flexible tube 42, including stiffening element 62 therein, fully inserted into a subject's vein 22 and, there is seen in FIG. 11, stiffener 62 slidably withdrawn from tube 42 utilizing cover cap hub 64.

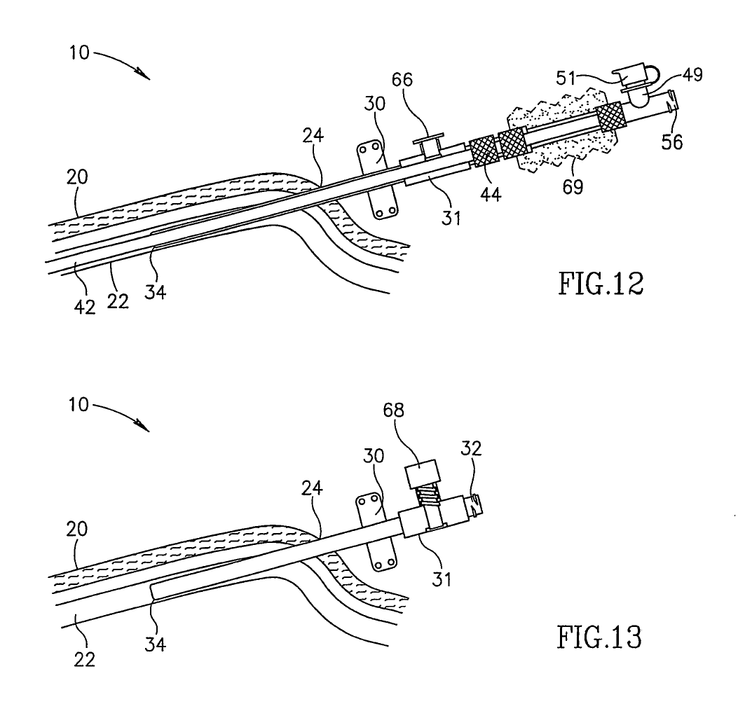

In accordance with a further embodiment of the present invention, referring to FIG. 12, multi-use entry-port 10 is formed having a locking device referenced 66 thereby to fasten catheter tube 42 at a predetermined position, relative to multi-use entry-port 10 and to the subject's vein 22. Sheath 48 (FIG. 3) is seen in a partially collapsed configuration, referenced 69, thereby to maintain sterility of the uninserted portion of tube 42, that is, the portion remaining external to entry port 10. In accordance with another embodiment, referring now to FIG. 13, multi-use entry-port 10 has a valve referenced 68 formed thereto, thereby to control or stop the rate of flow of liquid through catheter tube 42.

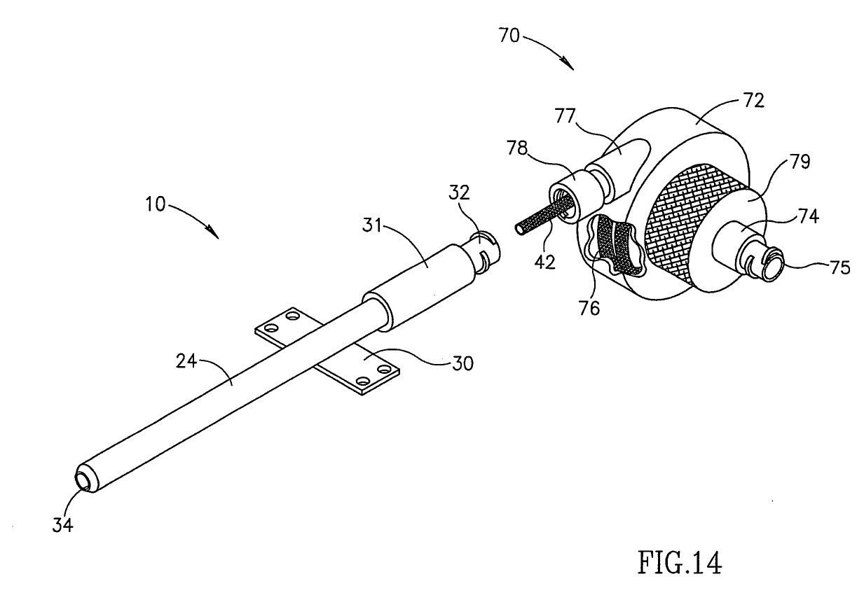

With regard to maintaining catheter tube 42 in a sterile environment prior to and during use, in accordance with a variation in an embodiment of the present invention, there is an integral sterile environment container, alternative to sterile sheath 48 (FIG. 3). Referring now to FIG. 14, there is seen a generally cylindrical sterile container, generally referenced 70, including a casing referenced 72, an inlet port referenced 74, having a connector 75, axially disposed with respect to casing 72. There is seen a tangentially disposed outlet port referenced 77 having a Luer Lock and a connector referenced 78 thereby to connect port 77 to connector 32 of entry-port 10. Catheter tube 42 is coiled reference 76 within casing 72. After connector 77 is attached to entry-port 10 at adapter 32, catheter tube 42 is extended by reeling from container 70 utilizing a knurled handle referenced 79, thereby to pass through entry-port 10 into a vein (not shown) of a subject.

An added variation of this embodiment to the present invention includes a clutch device incorporated into container 70, thereby controlling the force exerted on extending catheter tube 42 using knurled handle 79. This reduces the risk of damaging or piercing vein 22 while inserting catheter tube 4 through entry port 10 into vein 22.

Sterile container 70 has advantages of being compact and easily handled by a medical professional even in non-ideal circumstances.

There are practical advantages to the above-mentioned apparatus and method of insertion of a flexible catheter tube into a subcutaneous vein of a subject. The initial stage for carrying out the method in accordance with the preferred embodiment of the present invention is substantially similar to that presently utilized in most hospitals, using a short catheter or intracatheter. A multi-use entry-port or intracatheter is inserted into a subcutaneous vein of the subject. This requires no stitching to a subject's limb to be fixed in position. Adhesive tape is a successful securing device. The preferred embodiment further teaches the insertion of the flexible catheter tube into and through the entry-port or intracatheter and into the vein and secured to the entry port.

Furthermore, should there be an occurrence of fever or thrombosis, the flexible catheter tube is removed from the intracatheter and a new catheter tube inserted in its stead. Both medical professional and subject are spared the trauma and time of re-inserting the intracatheter into another site. The removed catheter tube tip is sent for laboratory culture testing. In addition, should the subject experience any pain at the site of the tube tip due to phlebitis, another shorter or longer catheter tube is used to replace the troublesome tube without necessitating the re-insertion of the entry-port.

With regard to maintaining sterility of the equipment and the subject, the above-mentioned procedure has an advantage insofar as there is no requirement for a sterile field of application of the catheter tube. Because the flexible tube is supplied in an integral sterile container, insertion may be carried out under virtually any conditions, in the open and unaffected by environmental contamination. Furthermore, only one medical professional, with little additional training, is able to carry out this procedure, without any specific immobilization or trauma of the subject. The procedure offers a safe and convenient method for atraumatic administration of intravenous therapy.

The present invention, also, provides for insertion of catheters and other similar tubular devices through a multi-use entry port into various organs of the body. The entry port provides the medical professional with the means for repeatedly accessing an organ without having to repeatedly pierce the skin and organ wall of the subject. In addition, such access is achieved without requiring a sterile environment since each tubular device to be inserted is enclosed within an integral sterile container.

It will be appreciated by persons skilled in the art that the present invention is not limited by the drawings and description hereinabove presented. Rather, the invention is defined solely by the claims that follow.

1. An improved intravenous catheter system including
a) a multi-use entry-port element having first and second ends having a bore formed there-between, said port element configured for transcutaneous positioning such that said second end is brought into liquid flow communication with a vein of a subject; and
b) a catheter having first and second ends and a flexible catheter-tube there-between, said catheter tube having a predetermined length and a diameter adapted for slidable insertion through said entry-port element into the vein of the subject.

2. An improved intravenous catheter system according to claim 1 wherein said multi-use entry-port element includes
a) a hub having a slide adapter-connector fixably disposed at said first end of said entry-port element, thereby to provide sealed slidable access of said catheter-tube into said entry-port element and thereafter into the vein of the subject;
b) a cannula having an aperture formed at a second end thereof, said cannula fixably attached to said hub and disposed at said second end of said entry-port element, said cannula adapted for insertion into the vein of the subject; and
c) a removable needle having first and second ends having a needle-hub attached at said first end, having a sharp extremity at said second end and having a length sufficient to extend through said entry-port element, said needle slidably housed in said entry-port, said sharp extremity projecting beyond said aperture at said second end of said cannula thereby to pierce through the skin and vein wall into the vein of the subject, and thereby to provide entry into the vein for said cannula.

3. An improved intravenous catheter system according to claim 2 wherein said entry-port element includes mounting lugs fixably disposed thereto, thereby to secure said entry-port element to the subject.

4. An improved intravenous catheter system according to claim 2 wherein said second end of said cannula is formed with a taper, thereby to provide a compression lip seal between said cannula aperture and said catheter tube.

5. An improved intravenous catheter system according to claim 2 wherein said slide adapter-connector includes a Luer Lock.

6. An improved intravenous catheter system according to claim 1 wherein said catheter includes
a) a connector element disposed at said first end of said catheter and having a removable cap, said connector element configured to facilitate, in the absence of said cap, connection of an intravenous therapeutic device to said first end of said catheter;
b) a slidable-connector element disposed at said second end of said catheter and having a removable cap, said slidable-connector element, configured to facilitate connection of said second end of said catheter to said first end of said entry-port element, thereby to facilitate sliding said catheter tube therethrough into said entry-port element and thereafter into the vein of the subject.

7. An improved intravenous catheter system according to claim 6 wherein said connector element includes a Luer Lock.

8. An improved intravenous catheter system according to claim 1, and also including an integral sterile environment containment element for enclosing said catheter tube within a sterile environment.

9. An improved intravenous catheter system according to claim 8 wherein said integral sterile environment containment element is selected from the group which consists of:
a) a longitudinal collapsible sheath configured to contain said catheter tube longitudinally therewithin; and
b) a cylindrical casing configured to contain said catheter tube as a withdrawable coil therewithin.

10. An improved intravenous catheter system according to claim 9 wherein said cylindrical casing includes a clutch device thereby to control forceful insertion of said catheter tube through said entry port into a vein, so as to avoid damaging the vein wall.

11. An improved intravenous catheter system according to claim 1, and also including a removable stiffener element slidably disposed within said flexible catheter tube, thereby to increase the stiffness of said catheter tube, and thereby to aid insertion thereof through said entry-port element into the vein of the subject.

12. An improved intravenous catheter system according to claim 1 wherein said entry-port element includes a selectably operable locking device for locking said catheter tube in a selected position with respect to a selected drug delivery location within the vein of the subject.

13. An improved intravenous catheter system according to claim 1 wherein said entry-port element includes a valve for regulating a flow of liquid through said catheter tube.

14. For use with an intravenous cannula element having first and second ends and having a bore formed therebetween, said cannula element configured for transcutaneous positioning such that said first end is adapted to protrude from a limb of a subject and said second end is brought into communication with an interior of a body organ of a subject, a self-contained sterile catheter apparatus which includes
a) first and second ends and a flexible catheter tube therebetween, said catheter tube having a predetermined length and a diameter adapted for slidable insertion through said bore of said intravenous cannula element into the body organ of the subject, and
b) an integral sterile environment containment element thereby to allow insertion of said catheter tube through said cannula element into the body organ of a subject in a generally non-sterile environment.

15. For use with an intravenous cannula element according to claim 14 wherein said catheter includes
a) a connector element disposed at said first end of said catheter and having a removable cap, said connector element configured to facilitate, in the absence of said cap, connection of an intravenous therapeutic device to said first end of said catheter;
b) a slidable-connector element disposed at said second end of said catheter and having a removable cap, said slidable-connector element, configured to facilitate connection of said second end of said catheter to said first end of said entry-port element, thereby to facilitate sliding said catheter tube therethrough into said entry-port element and thereafter into the vein of the subject.

16. For use with an intravenous cannula element according to claim 14 wherein said integral sterile environment containment element is selected from the group which consists of:
a) a longitudinal collapsible sheath configured to contain said catheter tube longitudinally therewithin; and
b) a cylindrical casing configured to contain said catheter tube as a withdrawable coil therewithin.

17. For use with an intravenous cannula element according to claim 14 and also including a removable stiffener element slidably disposed within said flexible catheter tube, thereby to increase the stiffness of said catheter tube, and thereby to aid insertion thereof through said entry-port element into the vein of the subject.

18. A method for introducing an improved intravenous catheter system into a subcutaneous vein of a subject, said method including the steps of:
a) introducing a multi-use entry-port, having a removable piercing-needle slidably housed therein, into an outer wall of a subcutaneous vein of a subject;
b) withdrawing the removable piercing-needle from the entry-port;
c) advancing the multi-use entry-port into the vein;
d) connecting a catheter device, having a flexible catheter tube, to the entry-port; and
e) slidably inserting the flexible catheter-tube through the entry-port into the subcutaneous vein of the subject.

19. The method according to claim 18 wherein said step of introducing includes the step of securing the entry-port element to the skin of the subject.

20. The method according to claim 18 wherein withdrawing the removable piercing-needle from the entry-port includes the step of partially withdrawing the removable piercing-needle from the entry-port, to guard the piercing needle and to avoid transfixing the vein.

21. A method according to claim 18, wherein, using a catheter tube having a removable stiffener element slidably disposed therein, includes an additional step of slidably removing the stiffener element from the catheter.
